(19) United States
(12) Patent Application Publication
(10) Pub. No.: US 2020/0107775 A1
(43) Pub. Date: Apr. 9, 2020

(54) Methods and Systems for Monitoring Sleep Apnea

(71) Applicant: Spacelabs Healthcare L.L.C., Snoqualmie, WA (US)

(72) Inventors: Philip de Chazal, Glenbrook (AU); Hakimeh Sadat Nadi Sadr Lahijany, Forest Lodge (AU)

(21) Appl. No.: 16/589,467

(22) Filed: Oct. 1, 2019

Related U.S. Application Data
(60) Provisional application No. 62/740,198, filed on Oct. 2, 2018.

(51) Int. Cl. A61B 5/00; A61B 5/0468; A61B 5/04; A61B 5/08

(52) U.S. Cl. CPC A61B 5/4818; A61B 5/7264; A61B 5/08; A61B 5/04014; A61B 5/0468

(57) ABSTRACT

Methods and systems for detecting and diagnosing sleep apnea include using three lead electrocardiogram (ECG) monitoring devices to calculate ECG derived respiration data. Beat-typing information from the ECG device's analyser is used to enhance the derived respiration data by removing beats identified as non-normal. Surrounding epoch information is integrated early and epoch posterior probabilities are thresholded in order to remove diffident epochs. The system is further trained as new data is collected in the database.

### CROSS-REFERENCE

The present specification relies on, for priority, U.S. Patent Provisional Application No. 62/740,198, entitled “Methods and Systems for Monitoring Sleep Apnea”, and filed on Oct. 2, 2018. The above-mentioned application is herein incorporated by reference in its entirety.

### FIELD

The present specification relates generally to monitoring health-related parameters and more specifically to methods and systems for detecting and diagnosing sleep apnea.

### BACKGROUND

Obstructive Sleep Apnea Syndrome (OSAS) is a condition characterized by pauses in breathing during sleep. The interruptions in breathing are typically caused by partial or complete blockage of the upper airway. OSAS is a sleep disorder that affects an estimated 5 to 15% of the general population. OSAS influences both the quality and the life expectancy of an untreated patient and often leads to daytime sleepiness and an increased risk for cardiovascular diseases. It is known that patients with OSAS are more likely to have high blood pressure, which is a major risk factor for heart disease and other cardiovascular conditions. Additionally, patients with OSAS are at a greater risk of developing Atrial Fibrillation (AFib) which is a condition characterized by an irregular, often rapid heartbeat, which can increase the risk of stroke, heart failure, and other complications. Although OSAS is a respiratory event, it can affect the systems in the body, especially the cardiovascular system. Therefore, electrocardiograms (ECG) can provide very valuable information about apneic events.

A standard diagnostic used to determine the presence or extent of OSAS is night-time polysomnography (PSG), a sleep test consisting of an overnight measurement session during which several physiological signals are recorded. The diagnostic power of PSG is counterbalanced by several drawbacks including cost, its obtrusiveness, and the impossibility to use it for long-term monitoring to better characterize OSAS. In the last two decades, there has been a trend to replace or complement PSG in OSAS monitoring with more portable and cost-effective tests. Sleep monitoring devices using wearable cardiac monitoring consumer devices such as an ECG patch and/or a PPG-based smartwatch are increasingly becoming popular.

The common principle behind the use of these devices is that cardiac activity is affected by the presence of apneic events. However, apneic events may not be the only phenomena influencing cardiac activity during sleep. For instance, many parasomnia events, such as limb movements and sleep terrors, are associated or followed by an arousal, just as most apneic events. Arousals associated with non-apneic events can degrade the performance of apneic event detection algorithms and hence their presence, and distinguishing between them, can be crucial in databases used for development of apnea-related algorithms and features.

The development of algorithms using cardiovascular features for OSAS monitoring has been an extensively researched topic in the last two decades. Several studies regarding automatic apneic event classification using ECG-derived features are based on the public Apnea-ECG database available on PhysioNet. One piece of data contained in these databases include epoch information, relating to time periods in which an apnea event may occur. Although this database is an excellent starting point for apnea topic investigations, it has been observed that algorithms for apneic-epochs classification that are successfully trained on this database (sensitivity>85%, false detection rate<20%) perform poorly (sensitivity<55%, false detection rate>40%) in other databases that may include patients with a broader spectrum of apneic events and sleep disorders. The reduced performance may be related to the complexity of breathing events, the increased number of non-breathing related sleep events, and the presence of non-OSAS sleep pathologies.

In addition, most of the Apnea-ECG databases do not have annotations for non-apneic events, which are described above. Further, the control groups are composed of healthy subjects, indicating that patients suffering from other disorders are not included. In addition to this, the Apnea-ECG databases typically do not include more complex apneic cases, such as subjects suffering from central apnea, or other sleep-related disorders, or sleep comorbidities, such as insomnia. The absence of these cases can limit the applicability of algorithms exclusively developed on the Apnea-ECG databases in real-world situations, such as for screening. As a consequence, even though the Apnea-ECG databases have allowed a common testing ground to compare the performance of different algorithms, its usage as the only data source obscures the limitations and generalizability of apneic epoch classification solutions. For instance, Lado et al. showed that a threshold-based classification on a single inter-beat-interval low-to-high frequency band power ratio [heart rate variability (HRV) feature] produces unreliable results in cases where the training and testing are performed between different databases. These differences could be related to the fact that a single feature may be insufficient to describe a complex phenomenon such as OSAS and the physiological differences between the different types of apneas and hypopneas. There is a need for improved features and algorithms that automatically characterize OSAS using cardiac signals, such as those derived from PSG and ECG monitors such as 3-lead monitors.

### SUMMARY

The following embodiments and aspects thereof are described and illustrated in conjunction with systems, tools and methods, which are meant to be exemplary and illustrative, not limiting in scope.

The present specification discloses a method for detecting and diagnosing sleep apnea in a patient, comprising: obtaining at least one signal, wherein the at least one signal is acquired and transmitted by at least one lead of an ECG monitoring device; processing the at least one signal; dividing the at least one signal into epochs; determining, from the at least one signal, RR interval data for each of the epochs, electrocardiogram-derived respiration (EDR) data for each of the epochs, and cardiopulmonary coupling (CPC) data for each of the epochs; extracting features from the RR interval data, the EDR data and the CPC data; combining the extracted features;

determining a first value from the combined extracted features for each epoch; applying a threshold to the determined first value to determine a classification of the first value for each epoch; and generating a value indicative of an extent or severity of the patient's sleep apnea based on the classifications of the first values for each of the epochs to assist a user in diagnosing sleep apnea.

Optionally, the classification comprises at least normal, unknown, and sleep disordered breathing.

Optionally, the value indicative of the extent or severity of the patient's sleep apnea is an apnea-hypopnea index (AHI). In optional embodiments, the first value is indicative of an epoch likelihood.

Optionally, obtaining the at least one signal comprises obtaining beat types.

Optionally, the method further comprises identifying the beat types which do not match at least one of a shape or a beat-to-beat timing of the patient's normal sinus rhythm beats and removing said identified beat types.

Optionally, the processing the at least one signal comprises: removing a baseline from each of the at least one signal to obtain a baseline wander; subtracting the baseline wander from the at least one signal to obtain a baseline wander free ECG signal; and determining RR intervals from the baseline wander free ECG signal.

Optionally, removing the baseline comprises using a first median filter and a second median filter.

Optionally, the method further comprises removing P waves from the at least one signal using a median filter.

Optionally, the method further comprises removing T waves from the at least one signal using a median filter.

Optionally, the method further comprises calculating a plurality of areas enclosed by QRS complexes, wherein each of said plurality of areas corresponds to each of the at least one leads of the ECG monitoring device; and summing said plurality of areas to obtain a pooled area for the ECG monitoring device.

Optionally, the determining the first value comprises determining a probability of that sleep apnea occurred in the epoch corresponding to that first value.

Optionally, the determining the probability comprises using data from multiple patients stored in a database.

Optionally, the method further comprises using the RR interval data and the EDR data to extract features from the CPC data.

The present specification also discloses a system for detecting and diagnosing sleep apnea in a patient, comprising: an electrocardiogram (ECG) device comprising at least one lead configured to obtain at least one signal from the patient; and at least one processor in communication with the ECG device configured to: process the at least one signal; divide the at least one signal into epochs; determine, from the at least one signal, RR interval data for each of the epochs, electrocardiogram-derived respiration data (EDR) for each of the epochs, and cardiopulmonary coupling (CPC) data for each of the epochs; extract features from the RR interval data, the EDR data and the CPC data; combine the extracted features; determine a first value from the combined extracted features for each epoch; apply a threshold to the determined first value to determine a classification of the first value; and generate a value indicative of an extent or severity of the patient's sleep apnea based on the classifications of the first values for each of the epochs to assist a user in diagnosing sleep apnea in the patient.

Optionally, the ECG device comprises at least three ECG leads.

Optionally, the classification is at least normal, unknown, and sleep disordered breathing.

Optionally, the value indicative of the extent or severity of the patient's sleep apnea is an apnea-hypopnea index (AHI).

Optionally, the first value is indicative of an epoch likelihood.

Optionally, the at least one processor is further configured to obtain beat types, identify the beat types which do not match at least one of a shape or a beat-to-beat timing of the patient's normal sinus rhythm beats, and remove said identified beat types.

Optionally, the at least one processor is further configured to remove a baseline from the at least one signal to obtain a baseline wander, subtract the baseline wander from the at least one signal to obtain a baseline wander free ECG signal, and determine RR intervals from the baseline wander free ECG signal.

Optionally, the system further comprises a first filter and a second filter configured to remove the baseline.

Optionally, the system further comprises a filter configured to remove at least one of P waves or T waves from the at least one signal.

Optionally, the at least one processor is further configured to use the RR interval data and the EDR data to extract features from the CPC data.

The present specification also discloses a method for detecting and diagnosing sleep apnea in a patient from at least one signal acquired from electrocardiogram device, wherein the at least one signal comprises a plurality of time periods, comprising: determining RR interval data, EDR data, and CPC data from the at least one signal for each of the plurality of time periods; extracting features from the RR interval data, the EDR data and the CPC data; combining the extracted features for each of the plurality of time periods; for each of the plurality of time periods, determining a value indicative of a probability that sleep apnea occurred from the combined extracted features; processing values indicative of the probability that sleep apnea occurred in each of the plurality of time periods to determine a degree of certainty; and generating an apnea-hypopnea index (AHI) based on the degree of certainty to assist a user in diagnosing sleep apnea in the patient. Optionally, each of the plurality of time periods comprise an epoch.

The aforementioned and other embodiments of the present shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1 illustrates an exemplary system environment for monitoring Electrocardiography (ECG) heart activity;

FIG. 2A is a graph of sensitivity versus specificity indicating performance of a known method of distinguishing a 60 second epoch of disorder-free breathing from epochs with disordered breathing;

FIG. 2B is a graph of sensitivity versus specificity indicating performance of a known method of distinguishing a patient with normal AHI from patients with AHI above 5, using the records;

FIG. 3 is a graph illustrating that the higher the threshold the greater the accuracy of the classifier in classifying the epoch, in accordance with embodiments of the present specification;

FIG. 4 is a flow chart illustrating a process of detecting and diagnosing sleep apnea, in accordance with some embodiments of the present specification;

FIG. 5A illustrates a graph of sensitivity versus specificity indicating performance of a method of distinguishing a 60 second epoch of disorder-free breathing from epochs with disordered breathing, in accordance with embodiments of the present specification;

FIG. 5B is a graph of sensitivity versus specificity indicating performance of distinguishing a patient with normal AHI from patients with AHI above 5, using the records, in accordance with embodiments of the present specification; and

FIG. 6 is an exemplary graphical user interface in accordance with embodiments of the present specification.

### DETAILED DESCRIPTION

In various embodiments, the present specification provides methods and systems to detect and diagnose sleep apnea. Electrocardiogram (ECG)-derived respiration data (EDR) is calculated using 3-lead ECG devices. Beat-typing information from the ECG device's analyser is used to enhance the EDR by removing beats identified as non-normal. In embodiments, surrounding epoch information is integrated early, and epoch posterior probabilities are thresholded in order to remove diffident epochs. An epoch posterior probability is defined as the application of posterior probability statistical analysis to epoch data or, stated differently, the conditional probability of a predefined event, such as an apneic event, determined after accounting for certain epoch data. In embodiments, the methods of the present specification are further trained as new data is collected in a database.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

It should be understood by those of skill in the art that any ECG monitor may be implemented in embodiments of the present specification. In embodiments, while not a requirement, it may be preferred that an ECG monitor having a small footprint, such as an ambulatory ECG recorder is used to acquire data for the systems of the present specification. In embodiments, but not limited to such embodiment, the ECG device may be a Holter recorder. In embodiments, the ECG recorder device may be embodied in a patch. In embodiments, the ECG recorder device employed may be in the form of a device where the electrodes and associated electronics are housed within a patch that is affixed to a patient. In embodiments described below where reference is made to a Holter 3 lead device, it should be understood by those of ordinary skill in the art that the reference is exemplary and not meant to be limiting.

Definitions

Beat-to-beat intervals reflects the time between heart beats and is usually measured in milliseconds (ms). Beat-to-beat intervals are also known as R-R intervals. R-R intervals may be derived from a physiological monitor capable of detecting consecutive heart beats such as but not limited to devices monitoring electrical activity, acoustic activity, blood pressure, blood velocity, blood oxygenation, heart movement and/or trans-thoracic impedance.

Heart Rate Variability (HRV) measures the specific changes in time, or variability, in R-R intervals.

Apnea is defined as the cessation of airflow through the upper airway for a period of ten seconds or longer. It is typically associated with a decline in blood oxygen saturation levels (desaturation).

Hypopnea is a reduction in airflow to less than 30% of normal airflow that leads to desaturation or an electrocortical arousal.

Apnea-Hypopnea Index (AHI) is at least one value that is indicative of at least one of an extent, frequency, and/or severity of sleep apnea. The AHI is preferably a function of the number of apnea and hypopnea events that occur per hour of sleep. Typically, the signal of interest is divided into epochs, a prediction is made for each epoch, and the number of epoch predictions per hour are determined and mapped to an AHI value. Some algorithms determine AHI where the apnea duration is scaled by the recording time, whereas some others determine AHI where the apnea duration is scaled by the sleep time. The AHI is calculated by dividing the number of apnea events by the number of hours of recording/sleep. While using the sleep time provides a slightly reduced error in estimating the AHI, the overall impact of selecting the AHI estimation approach on correctly identifying normal and apnoeic patients, is very negligible. The AHI values for adults are categorized as: Normal when AHI is less than 5, Mild when AHI is within a range of 5-15, Moderate when AHI is within a range of 15-30, and Severe when AHI is greater than 30.

Epoch refers to either overlapping or non-overlapping continuous divisions of time that the overall time of sleep is divided into. In some cases, an epoch is defined as a time period of approximately 30 seconds. In other cases, an epoch is defined as a time period of approximately 60 seconds. In still other cases, an epoch may be defined as a time period equal to any other length of time. In some embodiments of the present specification, acquired EDR data is monitored over 60 second epochs to determine, for each epoch, whether the data is apneic, or otherwise indicative of a patient experiencing apnea events.

A Record refers to a patient's data recorded at any one occasion. Records refers to the data for multiple patients recorded at more than one occasion for each patient. The data may encompass simultaneous records from an ECG Holter monitor and a Polysomnograph (PSG) from the patients.

Sensitivity refers to a proportion of true positive indications made by the detection systems and methods described in the present specification. A high sensitivity would reduce the likelihood of overlooking a possible positive indication of sleep apnea. It quantifies the avoidance of false negatives.

Specificity refers to a proportion of true negative indications made by the detection systems and methods described in the present specification. A high specificity would reduce the likelihood of overlooking a possible negative indication of sleep apnea. It quantifies the avoidance of false positives.

In one exemplary case, data collected from about 201 recordings of Polysomnograph (PSG) simultaneously with Holter 3-lead ECG device recordings, on 205 different patients, was used to create a sample database (wherein in four of the recordings were rendered unusable). The data may be generally acquired from a database. A sample database is used in the present specification to describe the various embodiments, their applications, and their relative performance as compared to some of the known methods and systems that are used to detect and diagnose sleep apnea. In embodiments, data from the PSG and from the Holter 3-lead ECG device was manually synchronized by aligning the QRS complexes of both sources. Based on the data, 40 patients were identified with normal AHI, 69 with mild AHI, 52 with moderate AHI, and 44 with severe AHI. Embodiments of the present specification can be used to estimate the likelihood of AHI with any other data collected for any set of patients.

Exemplary System Architecture

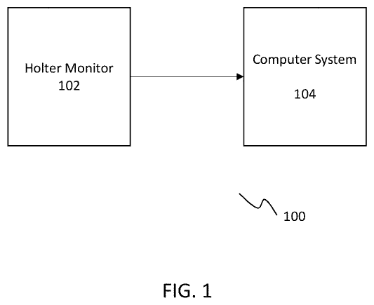

FIG. 1 illustrates an exemplary system 100 for monitoring Electrocardiography (ECG) heart activity. System 100 comprises an ECG monitor 102 and a computer system 104. Monitor 102 is a portable ECG monitoring device, such as a Holter monitor or patch, that may be worn by a patient over an extended period of multiple days, to enable continuous monitoring of cardiac activity of the patient.

Computer system 104 is in communication with monitor 102 and may include one or more processors (also interchangeably referred to herein as processors, processor(s), or processor for convenience), one or more storage devices, and/or other components. In one embodiment, computer system 104 is configured as a server (e.g., having one or more server blades, processors, and other server components as are known to those of skill in the art), a personal computer (e.g., a desktop computer, a laptop computer, and the like), a smartphone, a tablet computing device, and/or other device that is programmed to encode, decode, and/or analyse data as described herein. Processors may be programmed by one or more computer program instructions. Further, computer system 104 analyses the patient's cardiac condition by using the information collected by ECG monitor 102. In one embodiment computer system 104 is an analyser, known to persons of the art. In embodiments, computer system 104 is configured to execute one or more algorithms to process hundreds of thousands of beats seen over a multiple-day recording, which provides the clinician with comprehensive insight into ECG events and morphologies as well as ST segment, Heart Rate Variability (HRV) and QT analysis.

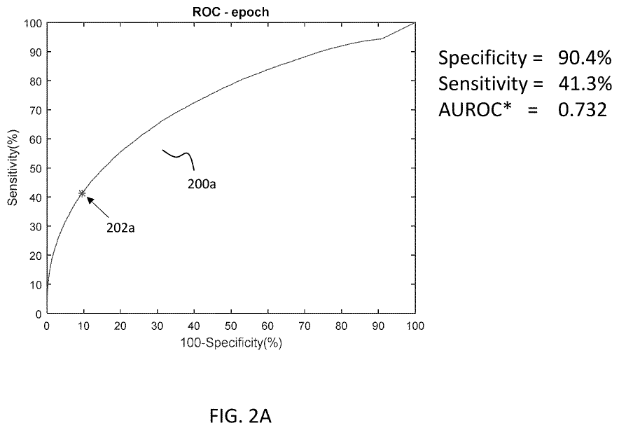

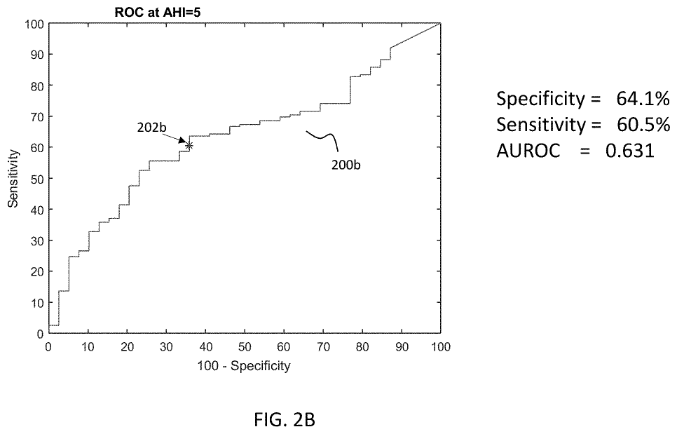

FIG. 2A is a graph 200a of sensitivity versus specificity indicating performance of a known method of distinguishing 60 second epoch free of disordered breathing from epochs with disordered breathing. The graph may be obtained using systems such as the system shown in FIG. 1. At a point 202a, the graph shows a specificity of 90.4% and a sensitivity of 41.3%. An Area Under the Receiver Operator Curve (AUROC) is 0.732. FIG. 2B is a graph 200b of sensitivity versus specificity indicating performance of a known method of distinguishing a patient with normal AHI from patients with AHI above 5, using the records. At a point 202b, the graph shows a specificity of 64.1% and a sensitivity of 60.5%, AUROC of 0.0631. The records encompass 201 CVS recordings, as stated earlier.

The above-described known methods of detecting and diagnosing sleep apnea use features based on heart rate variability (HRV) and surrogate respiratory effort, based on ECG derived respiration (EDR) which are obtained from the ECG signals using an ECG monitor. All features are obtained for non-overlapping windows of one minute in order to match the Apnea-ECG database epoch annotations.

Embodiments of the present specification provide several advantages over the existing methods and systems for detecting and diagnosing sleep apnea. One of the advantages is that the embodiments of the present specification detect and diagnose sleep apnea using features based on Cardiopulmonary Coupling (CPC), in addition to heart rate variability (HRV) and surrogate respiratory effort, based on ECG derived respiration (EDR) which are obtained from the ECG signals using an ECG monitor. In one embodiment, EDR is calculated using a 3-lead ECG device monitor. The data, specifically the beat data, collected by the ECG monitor is input into an ECG analyser, which is configured to enhance the EDR data by removing non-normal beats. The beat data is analysed to identify types of beats. Non-normal beats in the system are those which do not match the shape and/or beat-to-beat timing of the patient's normal sinus rhythm beats. Subsequently, the beats identified as non-normal are removed from further analysis. In embodiments, a baseline is removed from each ECG lead using two median filters. The first median filter has a width of 200 milliseconds (ms) and is designed to remove P waves and QRS complexes from the ECG. The second median filter has a width of 600 ms and is designed to remove T waves from the ECG. Once the P waves, QRS complexes, and T waves are removed from the ECG, the resulting signal is a baseline wander, which may be subtracted from the original ECG signal to provide a wander-free baseline ECG signal. For each detected QRS labelled as ‘N’ (normal beats) from the beat types, the area enclosed by the QRS complex and the isoelectric level is calculated. For QRSs labelled as other beat types the area is not calculated, as they are likely to have a different morphology (and therefore area) than the normal beats. Once the areas are calculated for all ‘N’ beats on the 3 ECG leads, they are summed together to give a pooled area which is used as the 3 lead EDR data. This signal is a surrogate for the chest respiratory effort signal.

In an embodiment, RR interval sequence for an epoch and EDR sequence for the same epoch are used to identify cardiopulmonary coupling features. Zeros are appended to the end of the RR and EDR sequences to form 256 point sequences. The average value is subtracted from each element of the sequence resulting in a new sequence. Each element in the new sequence then has an average of zero. Each element in the new sequence is then scaled by a constant factor so that sum of the squares of the new sequence elements is 1. The density is calculated by finding the discrete Fourier transform of sequence, squaring the transform element by element, and finding the absolute value of each element. Therefore, power-spectral density values in 32 bins ranged from 0 cycles/heartbeat interval to 0.5 cycles per heartbeat interval, are obtained.

In an embodiment, thresholds are set for a statistical posterior probability analysis of epoch data probabilities to remove diffident epochs, which refers to epochs with a high degree of uncertainty about the occurrence of sleep apnea. Classifier probability outputs for each epoch are used to apply thresholds to remove diffident epochs. The classifier is a linear system followed by a softmax stage linking the input features and the output probabilities. The system processes 3-lead ECG device data and provides a probability output for presence or absence of sleep disordered breathing in each epoch. The weights of the linear system are determined with the linear discriminant analysis calculation which determines the optimal (in Bayesian context) linear mapping for a classification system. The weights are determined from “training data”. In one embodiment, the training data is the usable ECG monitor recordings stated earlier, and associated apnea events labels determined from simultaneous overnight PSG recordings.

The output from the classifier is a probability estimate (e.g. with a value between 0 and 1) of apnea having occurred sometime during the epoch. Under un-thresholded conditions, when this value is less than 0.5, the epoch is labelled as “normal breathing” (NB) and when it is greater than or equal to 0.5 it is labelled as “sleep-disordered breathing” (SDB). It should be appreciated that the 0.5 threshold between NB and SDB may be re-labelled as any value. When the output is close to the threshold, i.e. 0.5, it indicates the classifier has some uncertainty in its decision. By choosing to only accept classifier outputs when the probability estimate is some distance away from 0.5 the classifier is forced to only make decisions on epochs it has some confidence in. For example, if a threshold of 0.1 is used, this means that any epoch with a value less than 0.4 would be labelled as “normal”, a value between 0.4 and 0.6 would be labelled as “unknown”, and greater than 0.6 labelled as “sleep-disordered breathing”. Accordingly, in some embodiments, the output is categorized, based on its value, as being at least one of NB, SDB, or unknown, where the value is NB if it's between a first value and a second value, the value is unknown if it's between the second value and a third value, and the value is SDB if it's between the third value and a fourth value, where the first value, second value, third value, and fourth value are sequential, and where the threshold divider between a NB category and SDB category is between the second value and third value.

In some embodiments, the present specification estimates an AHI from overnight ECG monitor device readings in a three-step process:

1) It estimates the probability of apnea having occurred in fix length epochs and after thresholding these probabilities labels the epochs as “normal breathing”, “unknown”, or “sleep-disordered breathing”, as described above.

2) In some embodiments, it sums the duration of the total epochs in an ECG recording labelled as “sleep-disordered breathing” (SDBTOTAL), divides this total duration value by an aggregate number of epochs labelled as either “normal breathing” or “sleep disordered breathing” (SDB), and then adjusts the resultant value by an appropriate factor to calculate an average “minutes per hour” of SDB for the recording. In some embodiments, it sums the number of epochs in an ECG recording labelled as “sleep-disordered breathing” (SDBTOTAL) and divides this value by the aggregate number of the epochs labelled as either “normal breathing” or “sleep disordered breathing” (SDB).

In some embodiments, the duration of epochs is summed and divided by a total number of hours of sleep time, which may be determined from sleep annotations, collected from ECG device readings. The obtained value is adjusted by a factor to calculate an average “minutes per hour” (MPH) of SDB for the sleep time. In a first embodiment, a duration of apnea and/or hypopnea per hour of recording is used to estimate the AHI, while in a second embodiment, duration of apnea and/or hypopnea per hour of sleep is used. In embodiments, either method may be used to estimate the AHI. In some cases, the minutes per hour of sleep method may result in less noise error than the minutes per hour of recording method. However, the impact of both methods to estimate AHI is minimal on the system's ability to discriminate between normal and apnea patients.

3) It scales the “minutes per hour” of SDB to form an estimated AHI. In embodiments, the scaling factor may range from 0.1 to 100 or any increment therein. In one embodiment, a scaling factor of 1.284 is used to estimate the AHI. In embodiments, AHI is estimated by adjusting the “minutes per hour” (MPH) figure by a pre-determined constant. In some embodiments a constant value of 0.5863 is used to optimise the performance of the system for discriminating patients at an AHI threshold of 5, and a constant value of 0.4300 is used to optimise the discriminating performance at AHI=15.

In alternative embodiments, AHI is estimated using other functions relating the AHI to the MPH, which use pre-determined constants (A, B, C), such as:

1) AHI=A+B*MPH

2) AHI=A+B*(MPH)^{C}

The values of A, B and C may be determined from training data.

Given that the basic engine of the system is a probabilistic model of the occurrence of apnea in an epoch, therefore thresholding the epoch probability outputs and removing the epochs with high degree of uncertainty (the diffident epochs) improves the per-epoch performance.

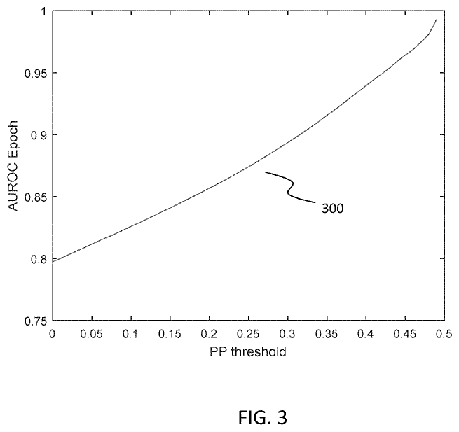

FIG. 3 is a graph 300 illustrating that the higher the threshold the greater the accuracy of the classifier in classifying the epoch, in accordance with embodiments of the present specification.

In one embodiment, feature values of each epoch are used for an early integration of surrounding epoch information, in order to obtain an expanded feature vector for each epoch.

Sleep disordered breathing not only affects the current epoch but also affects the epochs around it. For example, before an apnea event there maybe heart rate and breathing changes due to flow limitation, and after an apnea event there are recovery breaths and heart rate changes. Therefore, for each epoch, an expanded feature vector is formed by combining information from the surrounding epochs. For example, an expanded feature vector for epoch n can be formed by combining the feature information from feature vectors n−2, n−1, n, n+1 and n+2.

In one embodiment, a “supervised” learning machine learning approach is used to determine classifier parameters from feature vectors calculated from the provided training data. Embodiments of the present specification enable further training to determine new parameters for the classifier weights as new data is collected.

Embodiments of the present specification provide an improved method for detecting and diagnosing sleep apnea. In an embodiment, system elements of FIG. 1 are improved by the implementations of the present specification.

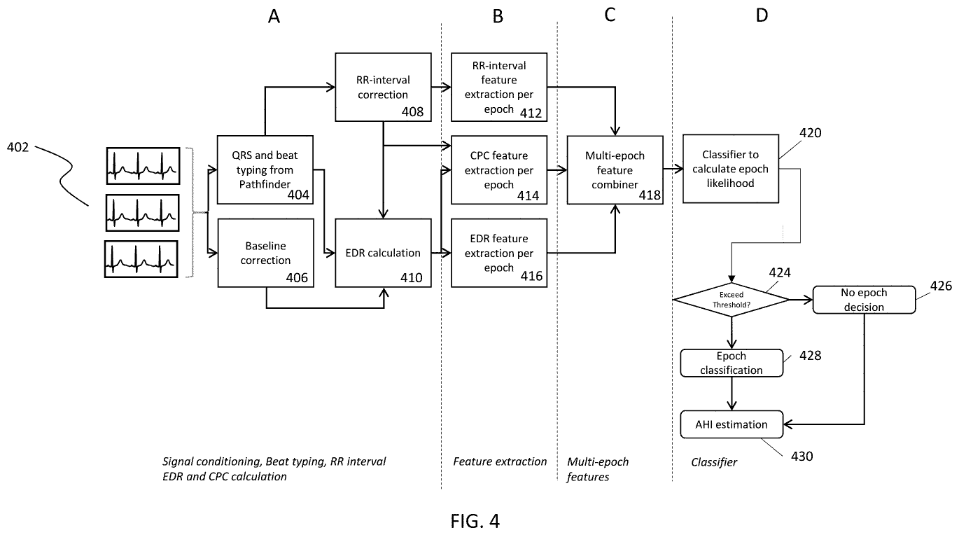

FIG. 4 is a flow chart illustrating a process of detecting and diagnosing sleep apnea, in accordance with some embodiments of the present specification. Embodiments illustrated in FIG. 4 are processed by an improved ECG monitoring device in accordance with the present specification. The process is broadly divided in to four sections—A, B, C, and D. Section A corresponds to the initial data collection, conditioning of signals, identifying beat types from the ECG data, and calculation of RR interval EDR and Cardiopulmonary Coupling (CPC) sleep spectrogram, in order to estimate a severity of apnea and/or hypopnea. Signals 402 from a 3-lead ECG monitoring device are transmitted to an analyzing system in accordance with embodiments of the present specification. In embodiments, beat type information is also made available to the system. Alternatively, the signals are used by the system to identify the beat types. At 404 and 406, the baseline is removed from each ECG lead using two median filters. In an embodiment, the first median filter has a width in a range of 100 ms to 300 ms and is designed to remove P waves and QRS complexes from the ECG. In one embodiment, the first median filter has a width of 200 ms. In one embodiment, the second median filter has a width in a range of 400 ms to 800 ms and is designed to knock out T waves from the ECG. In one embodiment, the second median filter has a width of 600 ms. Once all three waves are removed from the ECG, the resulting signal is the baseline wander, and this can then be subtracted from the original ECG to give a baseline wander free ECG signal. For each detected QRS labelled as ‘N’ from the beat types, the area enclosed by the QRS complex and the isoelectric level is calculated. For QRSs labelled as other beat types the area is not calculated, as they are likely to have a different morphology (and therefore area) than the normal beats. At 410, once the areas are calculated for all ‘N’ beats on the 3 ECG leads, they are summed together to yield a pooled area which is used as the 3 lead EDR. This signal is a single channel signal approximating the chest respiratory effort signal. Additionally, RR intervals are calculated and are optionally corrected at 408.

The resultant corrected signals are divided into epochs. Long epoch lengths may provide for a good estimate of features, whereas short epoch lengths may provide for better time resolution of apnea events. Therefore, an appropriate epoch length may be selected accordingly. In some embodiments, an epoch length of 60 seconds is used. In alternative embodiments, epoch lengths of ranging from 30 to 120 seconds, and any increment thereof, may be used. In alternative embodiments, epoch lengths of 30 or 120 seconds may be used.

Section B, corresponding to extraction of features, comprises extracting RR interval feature per epoch at 412. In some embodiments, ten time-domain RR features are included: the first five serial correlation coefficients corresponding to a delay of one to five RR-intervals; standard deviation of the RR intervals; standard deviation of the change in RR intervals; mean epoch RR interval; and two NN50 features. An interval-based RR-interval power spectral density (PSD) may be calculated to capture the heart rate variability (HRV). A sequence of RR intervals is associated with each epoch, where the index is beat number. The mean RR interval for that epoch is removed from each value, to yield a zero-mean sequence. The elements of this sequence are normalized by dividing each element by the square root of the sum of the squares of the sequence. This final step ensures the sum of the PSD is unity and that relative contributions of the frequency bands to the PSD are easily assessed. For epoch lengths of 60 seconds, the sequence is padded to 256. The Discrete Fourier Transform (DFT) is taken of the entire sequence. The DFT coefficients are squared to yield a periodogram estimate of the PSD.

At 414, CPC feature extraction is performed, and at 416, EDR feature extraction is performed for each epoch. In one embodiment, for CPC feature extraction, zeros are appended to the end of the RR and EDR sequences to form 256 point sequences (for 60 seconds epoch length). Similar to obtaining the RR interval PSD, the average value is subtracted from each sequence so that the sequence has zero average. Each element in the sequence is then scaled by a constant factor so that sum of the square of the sequence elements is 1. A cross density is calculated by finding the discrete Fourier transform of both sequences, multiplying the transforms together element by element, and finding the absolute value of each element. Subsequently, what is obtained is cross-spectral density values in 32 bins ranged from 0 cycles/heartbeat interval to 0.5 cycles per heartbeat interval. In embodiments, feature extraction using RR interval correction and calculation of EDR is performed simultaneously. Features extracted in section B are processed simultaneously at 418, by a multi-epoch feature combiner, in section C. For each epoch, an expanded feature vector is formed by combining information from the surrounding epochs. For example, an expanded feature vector for epoch n is formed by combining the feature information from feature vectors n−2, n−1, n, n+1 and n+2, by the combiner.

In section D, at 420, the combined multi-epoch features are input into a classifier, in order to obtain a classification for each epoch. In one embodiment, the classification comprises a label of one of the following: “normal breathing”, “unknown”, or “sleep disordered breathing”. An epoch likelihood is calculated. The output from the classifier is a probability estimate (with a value between 0 and 1) of apnea likely to have occurred sometime during the epoch. When this value is less than 0.5, the epoch is labelled as “normal breathing” and when it is greater than equal to 0.5 it is labelled as “sleep disordered breathing”. However, when this value is close to 0.5 it indicates the classifier has some uncertainty in its decision. By choosing to only accept classifier outputs when the probability estimate is some distance away from 0.5 the classifier is forced to only make decisions on epochs it has some confidence in. A threshold distance is pre-defined from the probability estimate that leads to uncertainty. In one embodiment, the threshold is defined as 0.1. In this case, any epoch with a classifier probability value less than 0.4 is labelled as “normal”, a value between 0.4 and 0.6 is labelled as “unknown”, and greater than 0.6 is labelled as “sleep disordered breathing”. At 424, the pre-defined threshold is used to determine whether an epoch is classified or is unknown. If the probability does not exceed the threshold, at 426, the system determines that no epoch decision can be made since the epoch was classified as “unknown”. However, if the threshold was exceeded, then at 428, the system obtains the epoch classification. At 430, all epoch classifications, including those classified as unknown', are used to estimate AHI.

In some embodiments, an AHI estimate is derived by first determining the average minutes-per-hour of apnoea from the predicted epoch annotations, then multiplying this figure by a predetermined constant. In the exemplary case illustrated herein, the predetermined constant was determined by first determining the number of apnoea minutes-per-hour of recording (for all the 201 CVS recordings). Second, the AHI value for each recording was used to label each recording as normal (AHI<5) or apnea (AHI>=5). Then, a receiver operator curve (ROC) was formed using the apnoea-minutes-per hour figures and the AHI determined labels. Following, the operating point on the ROC that resulted in a sensitivity of 80% was determined and associated apnoea minutes per hour determined (MPH_{80%sens}). Subsequently, the predetermined constant was then calculated as 5/MPH_{80%sens}. By using this methodology, the sensitivity of the system to was preset to 80%.

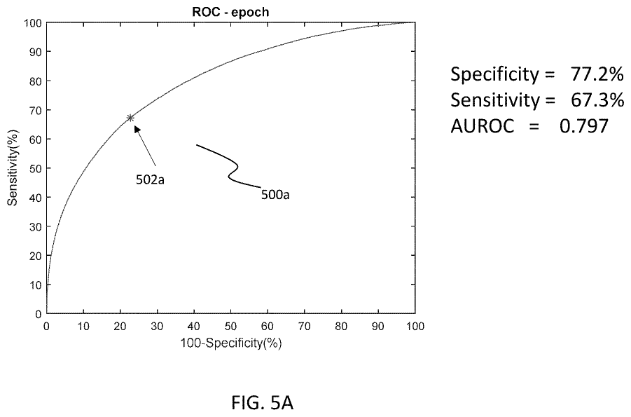

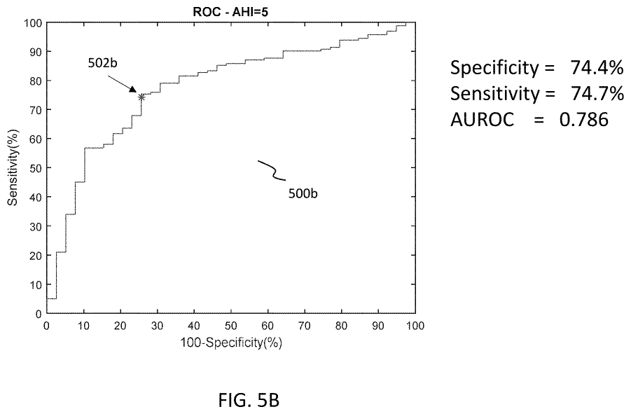

FIG. 5A illustrates a graph 500a of sensitivity versus specificity indicating performance of a method of distinguishing 60 second epoch free of disordered breathing from epochs with disordered breathing, in accordance with embodiments of the present specification. At a point 502a, the graph shows a specificity of 77.2% and a sensitivity of 67.3%, and AUROC is 0.797 which is an improvement over AUROC of 0.732 obtained using the prior art method and as illustrated in the graph of FIG. 2A. FIG. 5B is a graph 500b of sensitivity versus specificity indicating performance of distinguishing patient with normal AHI from patients with AHI above 5, using the records, in accordance with embodiments of the present specification. At a point 502b, the graph shows a specificity of 74.4% and a sensitivity of 74.7%, and AUROC of 0.786 which is an improvement of AUROC of 0.631 obtained using the prior art method and as illustrated in the graph of FIG. 2B.

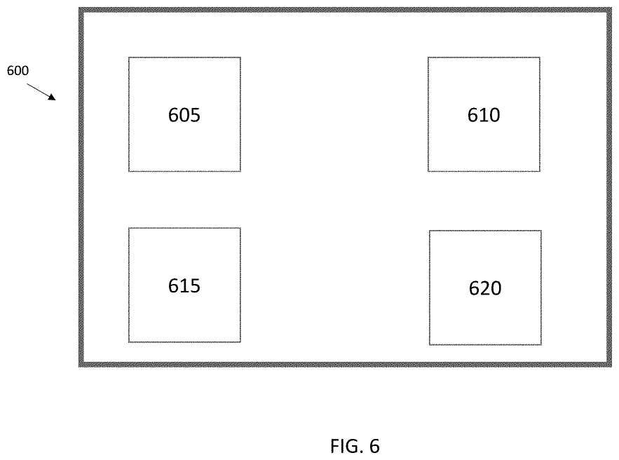

The output of various embodiments of the present specification is an AHI value for the patient, together with identification in the recording of areas which are suspected to show apnea. These are indicated on the report given to a physician and allow the physician to review the data and decide on follow-up investigation to be carried out. Accordingly, in one embodiment, referring to FIG. 6, the system generates data representative of a graphical user interface 600 and transmits it to a display device, such as a monitor, a mobile phone, a laptop, or tablet computer. The graphical user interface 600 may comprise a plurality of sections 605, 610, 615, 620 which may have one or more of the following visual representations: a) an indication of the AHI or any values related thereto, b) an explanation of the medical meaning of the AHI or any values related thereto, including a likelihood, extent, degree, or severity of the monitored patient's sleep apnea, c) graphs of any of the aforementioned values over time and/or sensitivity vs. specificity as described above, and/or d) recommendations for further testing or other action items.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A method for detecting and diagnosing sleep apnea in a patient, comprising:
obtaining at least one signal, wherein the at least one signal is acquired and transmitted by at least one lead of an ECG monitoring device;
processing the at least one signal;
dividing the at least one signal into epochs;
determining, from the at least one signal, RR interval data for each of the epochs, electrocardiogram-derived respiration (EDR) data for each of the epochs, and cardiopulmonary coupling (CPC) data for each of the epochs;
extracting features from the RR interval data, the EDR data and the CPC data;
combining the extracted features;
determining a first value from the combined extracted features for each epoch;
applying a threshold to the determined first value to determine a classification of the first value for each epoch; and
generating a value indicative of an extent or severity of the patient's sleep apnea based on the classifications of the first values for each of the epochs to assist a user in diagnosing sleep apnea.

2. The method of claim 1, wherein the classification comprises at least normal, unknown, and sleep disordered breathing.

3. The method of claim 1, wherein the value indicative of the extent or severity of the patient's sleep apnea is an apnea-hypopnea index (AHI).

4. The method of claim 1, wherein the first value is indicative of an epoch likelihood.

5. The method of claim 1, wherein obtaining the at least one signal comprises obtaining beat types.

6. The method of claim 5, further comprising identifying the beat types which do not match at least one of a shape or a beat-to-beat timing of the patient's normal sinus rhythm beats and removing said identified beat types.

7. The method of claim 1, wherein processing the at least one signal comprises:
removing a baseline from each of the at least one signal to obtain a baseline wander;
subtracting the baseline wander from the at least one signal to obtain a baseline wander free ECG signal; and
determining RR intervals from the baseline wander free ECG signal.

8. The method of claim 7, wherein removing the baseline comprises using a first median filter and a second median filter.

9. The method of claim 1, further comprising removing P waves from the at least one signal using a median filter.

10. The method of claim 1, further comprising removing T waves from the at least one signal using a median filter.

11. The method of claim 1, further comprising:
calculating a plurality of areas enclosed by QRS complexes, wherein each of said plurality of areas corresponds to each of the at least one leads of the ECG monitoring device; and
summing said plurality of areas to obtain a pooled area for the ECG monitoring device.

12. The method of claim 1, wherein the determining the first value comprises determining a probability of that sleep apnea occurred in the epoch corresponding to that first value.

13. The method of claim 12, wherein the determining the probability comprises using data from multiple patients stored in a database.

14. The method of claim 1, further comprising using the RR interval data and the EDR data to extract features from the CPC data.

15. A system for detecting and diagnosing sleep apnea in a patient, comprising:
an electrocardiogram (ECG) device comprising at least one lead configured to obtain at least one signal from the patient; and
at least one processor in communication with the ECG device configured to:
process the at least one signal;
divide the at least one signal into epochs;
determine, from the at least one signal, RR interval data for each of the epochs, electrocardiogram-derived respiration data (EDR) for each of the epochs, and cardiopulmonary coupling (CPC) data for each of the epochs;
extract features from the RR interval data, the EDR data and the CPC data;
combine the extracted features;
determine a first value from the combined extracted features for each epoch;
apply a threshold to the determined first value to determine a classification of the first value; and
generate a value indicative of an extent or severity of the patient's sleep apnea based on the classifications of the first values for each of the epochs to assist a user in diagnosing sleep apnea in the patient.

16. The system of claim 15, wherein the ECG device comprises at least three ECG leads.

17. The system of claim 15, wherein the classification is at least normal, unknown, and sleep disordered breathing.

18. The system of claim 15, wherein the value indicative of the extent or severity of the patient's sleep apnea is an apnea-hypopnea index (AHI).

19. The system of claim 15, wherein the first value is indicative of an epoch likelihood.

20. The system of claim 15, wherein the at least one processor is further configured to obtain beat types, identify the beat types which do not match at least one of a shape or a beat-to-beat timing of the patient's normal sinus rhythm beats, and remove said identified beat types.

21. The system of claim 15, wherein the at least one processor is further configured to remove a baseline from the at least one signal to obtain a baseline wander, subtract the baseline wander from the at least one signal to obtain a baseline wander free ECG signal, and determine RR intervals from the baseline wander free ECG signal.

22. The system of claim 21, further comprising a first filter and a second filter configured to remove the baseline.

23. The system of claim 21, further comprising a filter configured to remove at least one of P waves or T waves from the at least one signal.

24. The system of claim 15, wherein the at least one processor is further configured to use the RR interval data and the EDR data to extract features from the CPC data.

25. A method for detecting and diagnosing sleep apnea in a patient from at least one signal acquired from electrocardiogram device, wherein the at least one signal comprises a plurality of time periods, comprising:
determining RR interval data, EDR data, and CPC data from the at least one signal for each of the plurality of time periods;
extracting features from the RR interval data, the EDR data and the CPC data;
combining the extracted features for each of the plurality of time periods;
for each of the plurality of time periods, determining a value indicative of a probability that sleep apnea occurred from the combined extracted features;
processing values indicative of the probability that sleep apnea occurred in each of the plurality of time periods to determine a degree of certainty; and
generating an apnea-hypopnea index (AHI) based on the degree of certainty to assist a user in diagnosing sleep apnea in the patient.

26. The method of claim 25 wherein each of the plurality of time periods comprise an epoch.
